(19) United States
(12) Patent Application Publication
(10) Pub. No.: US 2007/0078409 A1
(43) Pub. Date: Apr. 5, 2007

(54) Disposable safety syringe guard and needle

(76) Inventor: Jeffrey I. Saltz, York, ME (US)

Correspondence Address: BOURQUE & ASSOCIATES;INTELLECTUAL PROPERTY ATTORNEYS, P.A., 835 HANOVER STREET, SUITE 301, MANCHESTER, NH, 03104, US

(21) Appl. No.: 11/242,752

(22) Filed: Oct. 4, 2005

(51) Int. Cl. A61M 5/32

(52) U.S. Cl. 604/198

(57) ABSTRACT

A protective needle guard assembly features a hub that is removably secured to a reusable syringes, a first and a second needle extending outwardly from the hub in opposite directions, and a moveable sheath that moves about an outer surface of the syringe. The needle guard includes an engagement device that engages the moveable sheath to secure the moveable sheath in an extended position wherein the moveable sheath extends beyond the distal end of the first needle. The moveable sheath features a retracted position wherein the first needle is substantially exposed. A cavity formed in the hub substantially covers and protects the second needle.

### TECHNICAL FIELD

The present invention relates to hypodermic syringes and more particularly, relates to reusable hypodermic syringes having a disposable safety syringe guard.

### BACKGROUND INFORMATION

There are two basic types of commonly used hypodermic syringes, i.e., disposable syringes and reusable syringes 1, FIG. 1. Disposable syringes are typically made from plastic and the needle is an integral component. In contrast, reusable syringes 1 typically include a two-part assembly. The first part features solid metal body 2 forming a cavity 3 for storage of the medicament and a plunger 4 for controlling the flow of the medicament. The second part features a removable needle tip assembly 5 that typically screws onto the distal end 6 of the metal body 2 opposite the plunger 4.

In recent years, disposable syringes have become increasingly popular due to their inherent convenience. However, disposable syringes do not provide the same feel as a reusable syringe due to their weaker and less rigid design. Moreover, many medical and dental procedures require aspirating the medicament with the recipient's blood prior to injection. This is procedure requires very precise control and feel of the plunger and is extremely difficult to do with a disposable syringe. As a result, several areas of the medical and dental profession still use reusable syringes 1.

It is well known within the medical and dental field that accidental needle scratching or puncture is a serious hazard and can result in the spread of disease to both medical and dental professionals as well as others. The known disposable needle assembles 5 for use with reusable syringes 1 feature a needle guard 7. The needle guard 7 typically features two guards sized and shaped to fit over the needles 8. In use, the needle guards 7 are removed by placing the user's hands on each of the needle guards 7 and pulling the needle guards 7 down the length of the needles 8. Once the needle guards 7 are removed, both needles 8 (i.e., the proximal 8′ and distal 8″) are exposed.

Unfortunately, the needle guard 7 design suffers from several limitations. One limitation of this known needle guard 7 design requires the user to place his/her hand in close proximity to the needles 8 when removing the needle guards 7. This increases the possibility of the user accidentally coming in contact with the needles 8.

Another limitation involves the reinstallation of the needle guards 7 after the reusable syringe 1 has been used. To reinstall the distal needle guard 7, the user again must place his hands on the needle guard 7 and then align the needle 8 with the opening in the needle guard 7. This again places the user's hand in close proximity to the needles 8. Moreover, the needle guard 7 on the proximate end 8′ cannot be reinstalled.

Yet a further problem with the known needle guard 7 design is that it is possible to damage the needles 8 during the removal or installation of the needle guards 7. As mentioned above, removal and reinstallation of the needle guards 7 require the needles 8 to be moved down the length of the bore of the needle guards 7. Should the needles 8 come in contact with the bore of the needle guards 7 during this movement of the needles 8, the tips of the needles 8 can be easily damaged creating a barb. This not only requires the needle assembly 5 to be replaced (further increasing the likelihood of an accident), but is wasteful, time consuming, and expensive.

A further limitation of the known needle guard 7 design is that the proximal needle 8′ is exposed once the needle guard 7 is removed. This exposed needle 8′ increases the possibly of the user being injured.

While there currently exists other safety needle designs, most of these designs are for disposable needles and are not compatible with reusable syringes 1. Additionally, the known safety needle designs are complex and generally either require the user to place his/her hands in close proximity to the needle or require expensive retraction mechanisms that are difficult to manufacture.

Accordingly, what is needed is a safety needle that works with reusable syringes 1. The safety needle should be inexpensive and easy to manufacture and should not require the user to place his/her hands near the needle during either removal or reinstallation. Additionally, the safety needle should minimize the chance of damaging the needle during either removal or reinstallation. Moreover, the safety needle should reduce the potential danger of both the proximal and distal ends of the needles.

It is important to note that the present invention is not intended to be limited to a system or method which must satisfy one or more of any stated objects or features of the invention. It is also important to note that the present invention is not limited to the preferred, exemplary, or primary embodiment(s) described herein. Modifications and substitutions by one of ordinary skill in the art are considered to be within the scope of the present invention, which is not to be limited except by the following claims.

### SUMMARY

According to one embodiment, the present invention features a safety needle assembly comprising a hub and a moveable sheath. The hub includes a first end having a first engagement device (preferably a threaded cavity) for removably engaging with a reusable syringe, a first needle extending outwardly from a second end, and at least one engagement device disposed about an outer surface of the hub. In the exemplary embodiment, the engagement device includes a biased tab, detent, or ball. The threaded cavity is preferably sized and shaped to substantially cover a second needle extending outwardly from the hub in substantially the opposite direction as the first needle.

The moveable sheath includes a generally tubular shaped body having an inner diameter sized and shaped to fit over the outer surface of the hub and an outer surface of the reusable syringe and at least one engagement element that acts with the at least one engagement device to position the moveable sheath in at least one predefined position. The moveable sheath is moveable along a longitudinal axis of the hub between an extended position whereby the moveable sheath is at least partially disposed about the hub and extends beyond a distal end of the first needle and a retracted position whereby the moveable sheath is substantially disposed about a barrel of the reusable syringe and the hub such that the first needle is substantially exposed.

According to one embodiment, the engagement element includes at least a first engagement element disposed proximate a distal end of the moveable sheath. The first engagement element positions the moveable sheath in the extended position and includes at least one aperture or recessed region. The engagement element may also include at least a second engagement element disposed proximate a proximal end of the moveable sheath. The second engagement element positions the moveable sheath in the retracted position and may include at least one recessed region or aperture.

The hub and the movable sheath optionally include a protrusion and a channel disposed along a longitudinal axis thereof. The protrusion and the channel define a predetermined path (either linear or helical) between the extended and the retracted positions along which the moveable sheath moves.

The safety needle assembly also may include an end device adapted to be disposed proximate a distal end of the moveable sheath. The end device substantially prevents access to the needle when the needle assembly is disposed in the extended position. According to one embodiment, the end device includes a cap. Alternatively, the end device includes a pierceable membrane.

According to another embodiment, the present invention features a needle comprising a reusable syringe and a needle guard assembly. The reusable syringe features a body portion defining an internal cavity sized and shape to contain a quantity of a medicament to be injected. A first fastener is disposed proximate a distal end of the body and a plunger disposed proximate a proximal end of the body. The plunger is adapted to control the flow of the medicament.

The needle guard assembly includes a hub having a first end having a second fastener for removably engaging with the first fastener of the reusable syringe, a needle extending outwardly from a second end, and at least one engagement device disposed about an outer surface of the hub. A moveable sheath is disposed about an outer surface of the hub and the reusable syringe and includes a generally tubular shaped body having an inner diameter sized and shaped to fit over the outer surface of the hub and an outer surface of the reusable syringe. At least one engagement element acts with the engagement device to position the moveable sheath in at least one predefined position. The moveable sheath is moveable along a longitudinal axis of the hub between an extended position whereby the moveable sheath is at least partially disposed about the hub and extends beyond a distal end of the needle and a retracted position whereby the moveable sheath is substantially disposed about a barrel of the reusable syringe and the hub such that the needle is substantially exposed.

### BRIEF DESCRIPTION OF THE DRAWINGS

These and other features and advantages of the present invention will be better understood by reading the following detailed description, taken together with the drawings wherein:

FIG. 1 is a plan view of a prior art reusable syringe and needle guard;

FIG. 2 is a perspective view of one embodiment of the disposable needle guard assembly shown in combination with a reusable syringe in an extended position accord to the present invention;

FIG. 3 is a perspective view of one embodiment of the disposable needle guard assembly shown in combination with a reusable syringe in a retracted position accord to the present invention;

FIG. 4 is perspective view of one embodiment of the disposable needle guard assembly according to the present invention;

FIG. 5 is a cross sectional view of the disposable needle guard assembly shown in FIG. 4 taken along line V;

FIG. 6 is a perspective view of one embodiment of the moveable sheath according to one embodiment of the present invention;

FIG. 7 is a cross sectional view of another embodiment of the moveable sheath according to one embodiment of the present invention; and

FIG. 8 is a cross section view of the moveable sheath shown in FIG. 7 taken along line VIII.

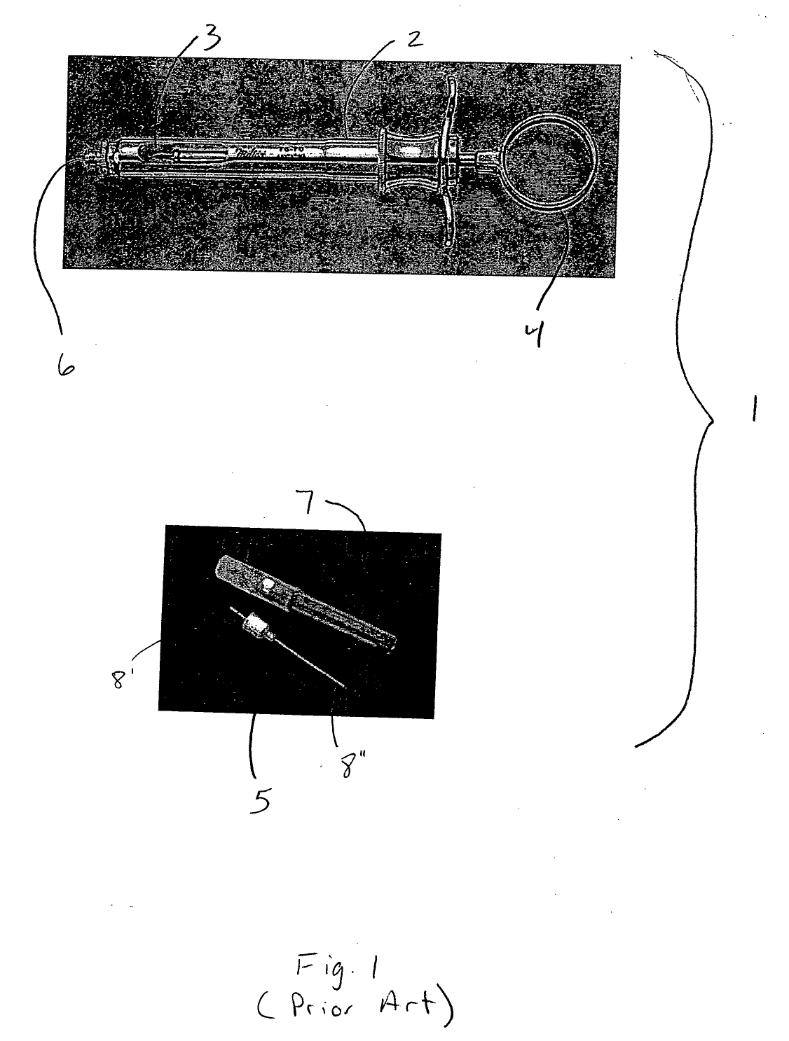

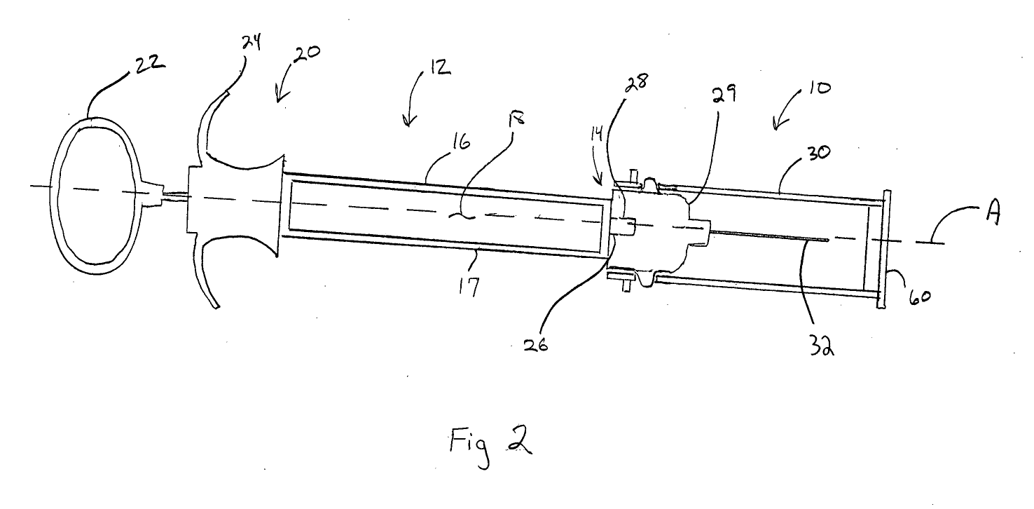

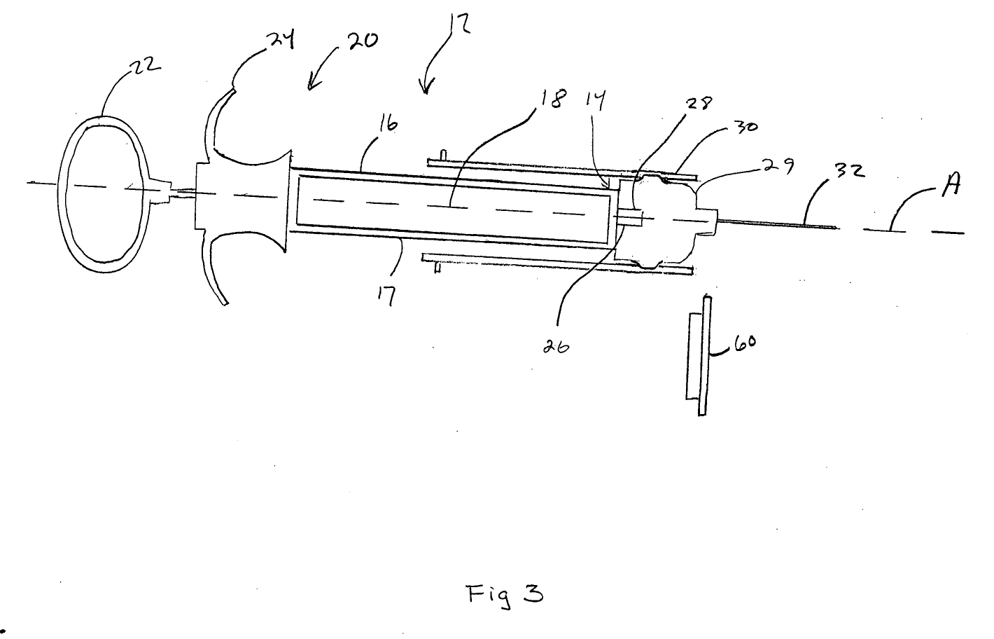

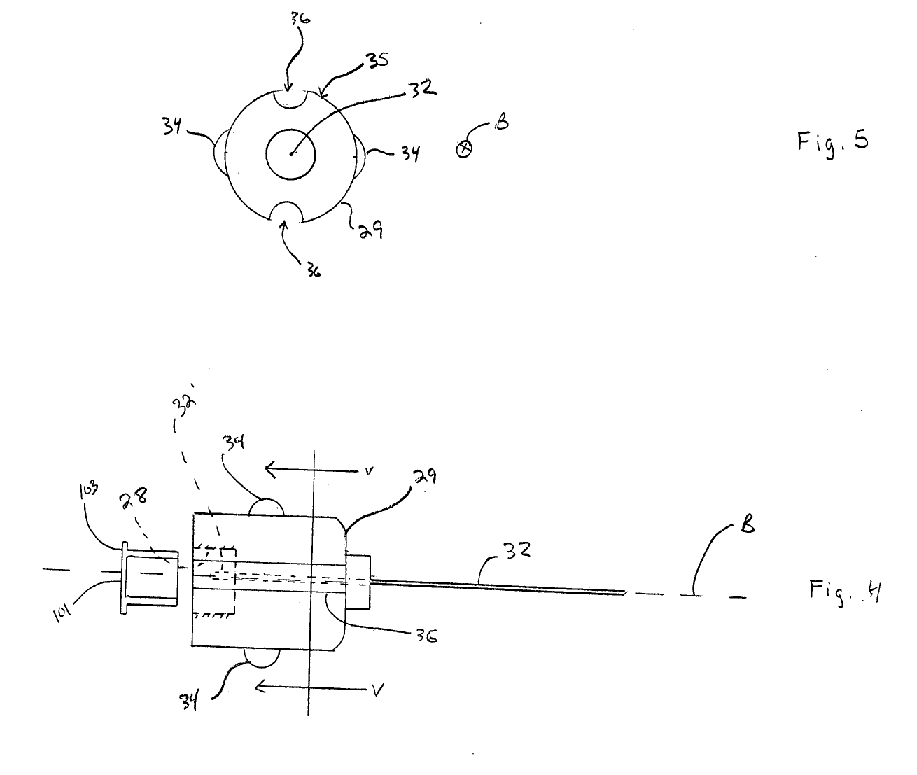

### DETAILED DESCRIPTION OF THE PREFERRED EMBODIMENTS

The present invention features a disposable needle guard assembly 10, FIGS. 2-3, for use with a reusable syringe 12. The disposable needle guard assembly 10 is removably secured to the distal end 14 of a standard reusable syringe 12 and is intended to be discarded after use whereas the reusable syringe 12 can be sterilized and reused. As will be explained in greater detail hereinbelow, the disposable needle guard assembly 10 according to the present invention features a moveable sheath or guard 30 that minimizes the likelihood of accidental contact with the needle 32.

As mentioned above, the reusable syringe 12 preferably features a traditional reusable syringe 12 having a body 16, preferably constructed primarily from metal, defining a cavity 18 with the barrel 17 sized and shaped to contain a quantity of a medicament. A first end 20 of the body 16 features a moveable plunger or piston 22 against which the user presses to control the flow of the medicament and one or more hand grips or flanges 24 that can be used by the user as leverage to aid in controlling the plunger 22 as is well known. The distal or second end 14 features a first engagement device 26, preferably an externally threaded male protrusion, for releasibly engaging with the disposable needle guard assembly 10.

The disposable needle guard assembly 10 features a hub 29 having needle 32 extending outwardly and a second engagement device 28 that engages with the first engagement device 26 in any manner known to those skilled in the art. In the preferred embodiment, the second engagement device 28 includes an internally threaded cavity or aperture that is sized and shaped to threadably accept the first engagement device 26.

According to the preferred embodiment, the tip of the proximal needle 32′ is disposed within the cavity 28. As a result, the cavity 28 forms a needle guard that reduces the likelihood of a user injuring himself/herself on the proximal needle 32′. Optionally, the present invention may feature a cap or plug 101 sized and shaped to engage the cavity 28 and cover the needle 32′ thereby further reducing the likelihood of injury and damage to the needle 32′ and preventing contamination of the needle 32′. The plug 101 may threadably engage the cavity 28 or alternatively may include a friction connection. A flanged region 103 may be provided on the plug 101 to aid in the removal of the plug 101.

The disposable needle guard assembly 10 also features a moveable sheath or guard 30 that minimizes or eliminates the likelihood of accidental contact with the needle 32. The moveable sheath or guard 30 includes a generally cylindrical, hollow tubular body having a inner diameter large preferably slightly larger than the outer diameter of the hub 29 of the disposable needle guard assembly 10 and barrel 17 of the reusable syringe 12. In use, the moveable sheath 30 moves along the longitudinal axis A of the disposable needle guard assembly 10 and reusable syringe 12 and includes a protected or extended position as shown in FIG. 2 and an in-use or retracted position as shown in FIG. 3. In the position shown in FIG. 2, the needle 32 is shielded by the moveable sheath 30 such that contact with the needle 32 is substantially prevented while in the position shown in FIG. 3, the needle 32 is substantially fully exposed such that the syringe functions normally.

Referring specifically to FIGS. 4 and 5, the hub 29 features at least one (preferably two or more) biased devices 34 that extend outwardly beyond the outer surface 35 of the hub 29. In the exemplary embodiment, the biased devices 34 feature a biased tabs. Alternatively, the biased devices 34 may include, but are not limited to, biased balls, biased clips, or the like. As will be explained in greater detail hereinbelow, the biased devices 34 engage the moveable sheath 30 in predetermined positions.

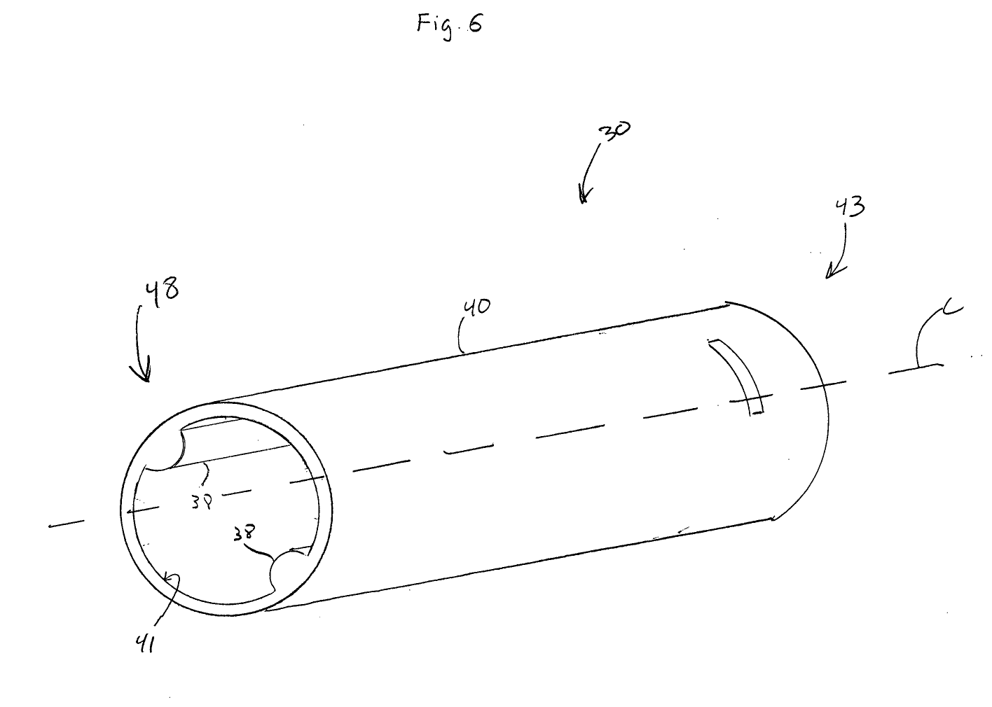

The hub 29 also optionally features at least one channel, slot, or groove 36 disposed proximate the outer surface 35 and generally along the longitudinal axis B of the hub. The channels 36 engage the protrusions 38 (FIG. 6) disposed along longitudinal axis C the moveable sheath 30 such that the moveable sheath 30 moves along a predefined track when moving between the extended and retracted positions. While the channels 36 have been shown as substantially linear, this is not a limitation of the present invention unless otherwise specifically recited as such. In an alternative embodiment, the channels 36 may be nonlinear (such as, but not limited to, a helical or spiral shape) such that the moveable sheath 30 rotates as it moves between the extended and retracted positions.

Referring specifically to FIGS. 6-8, the moveable sheath 30 features a hollow, generally cylindrical body 40 having a inner diameter large preferably slightly larger than the outer diameter of the hub 29 of the disposable needle guard assembly 10 and barrel 17 of the reusable syringe 12. In use, the moveable sheath 30 moves along the longitudinal axis A of the disposable needle guard assembly 10 and reusable syringe 12 and includes a protected or extended position as shown in FIG. 2 and an in-use or retracted position as shown in FIG. 3.

The body 40 of the moveable sheath 30 is preferably constructed from a generally rigid material such as, but not limited to, plastic of the like. The body 40 is preferably transparent or translucent such that the needle 32 may be at least partially seen while in the extended position. The inner surface 41 of the moveable sheath 30 preferably includes at least one protrusion 38. The protrusion 38 is sized and shaped to engage with the channel 36 of the hub 29 such that the moveable sheath 30 moves along a predefined track or path between the extended and retracted positions. Those skilled in the art will recognize that the orientation of the channel 36 and protrusion 38 may be reversed.

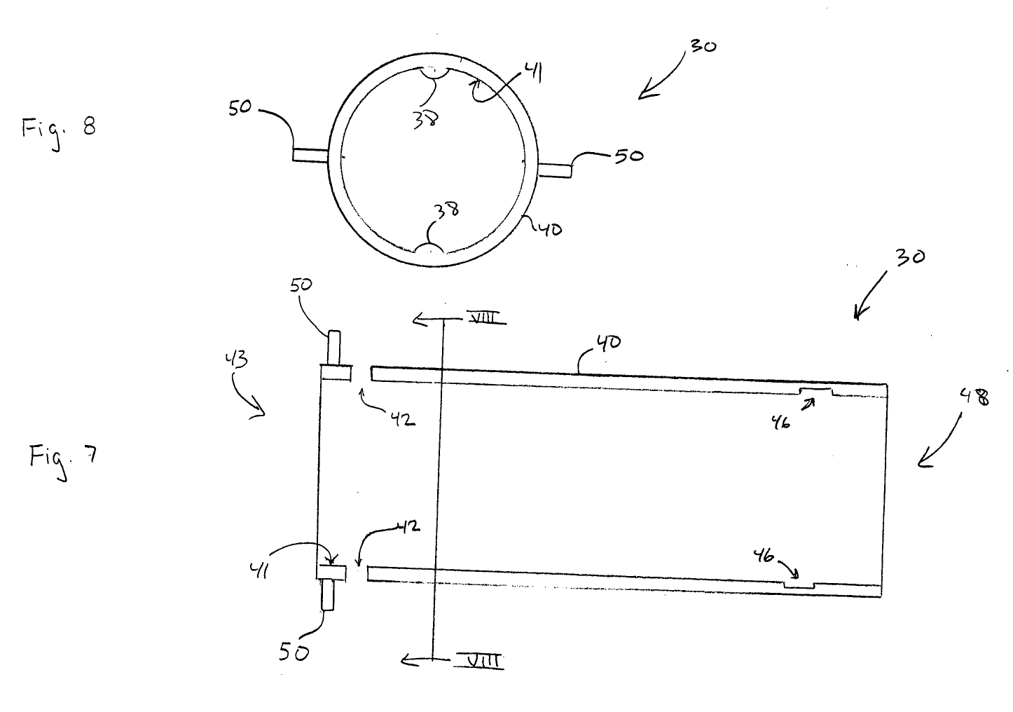

Referring specifically to FIG. 7, the moveable sheath 30 also features at least one engagement element 42, preferably two or more, disposed proximate a first or proximal end 43 of the moveable sheath 30. The engagement elements 42 act with the biased device 34 of the hub 29 to secure the moveable sheath 30 in the extended position. In the exemplary embodiment, the engagement elements 42 include an aperture or radially extending slot or groove sized and shaped such that the biased devices 34 (preferably biased tabs) are at least partially disposed within the apertures 42. With the biased tabs 34 disposed within the apertures 42, the position of the moveable sheath 30 is substantially locked in the extended position and the needle 32 is protected. To retract the moveable sheath 30, the biased tabs 34 are depressed and the moveable sheath 30 is now free to move.

In the exemplary embodiment, the engagement elements 42 feature an aperture that extends through the thickness of the body 40 of the moving sheath 30. Alternatively, the engagement elements 42 may also include a depressed region or cavity. According to this embodiment, when sufficient force is applied to the moveable sheath 30, the engagement devices 34 are released from the depressed region 42 and the moveable sheath 30 is free to move.

The body 40 of the moveable sheath 30 optionally includes at least one engagement element 46 disposed proximate the second or distal end 48 of the moveable sheath 30. These engagement elements 46 engage the engagement devices 34 to secure the moveable sheath 30 in the retracted position. In the exemplary embodiment, the engagement elements 46 feature depressed region or cavity as explained hereinabove.

Once in the retracted position, the engagement elements 46 preferably act with the engagement devices 34 to secure the moveable sheath 30 in the retracted position. To place the disposable needle guard assembly 10 from the retracted position to the extended position, the user simply places his/her hand proximate the first end 43 and urges the moveable sheath 30 towards the extended position. The moveable sheath 30 also optionally features one or more finger grips 50, preferably disposed proximate the first end 43, which facilitate moving the moveable sheath 30 between the extended and retracted positions.

The moveable sheath 30 also optionally features an end device 60, FIGS. 2 and 3. The end device 60 is secured to the distal end 48 of the moveable sheath and protects the needle 32 from accidental damage and also further prevents the user from accidentally coming in contact with the needle 32. According to one embodiment, the end device 60 includes a removable cap or plug that preferably frictionally engages the inner surface 41 of the moveable sheath 30. Alternatively, the cap or plug may thread onto the moveable sheath 30. The end device 60 may also include a membrane. In use, the membrane covers the distal end 48 of the moveable sheath 30. When the moveable sheath 30 is moved from the extended position to the retracted position, the needle 32 pierces the membrane.

It is important to note that the present invention does not require the user to place his/her hands near the needle 32 when moving the moveable sheath 30 between the extended and retracted positions. During movement of the moveable sheath 30, the user's hands are at all times disposed proximate the first end 43. This reduces the likelihood of accidental contact with the needle 32.

Additionally, the moveable sheath 30 can be placed in the retracted position prior to the needle 32 coming into contact with the patient thus allowing the user full and unobstructed sight of the tip of the needle 32. In contrast, some of the known needle guards require contact with the patient into order to retract the needle guard thereby reducing the accuracy of the injection.

The disposable needle guard assembly 10 according to the present invention is specifically designed to work with reusable syringes 12. As discussed above, the moveable sheath 30 moves along the outer surfaces of the hub 29 and the reusable syringe 12. In contrast, the guard of many on the known needle guards moves into a cavity disposed within the outer surface of needle and accordingly are not compatible with existing reusable syringes.

The disposable needle guard assembly 10 according to the present invention is inexpensive to manufacture. Many of the known needle guards require complex retraction mechanisms such as counter-springs and the like in order to retract the guard. This adds complexity and substantial cost to the manufacturing process. Because the disposable needle guard assembly 10 according to the present invention are disposable, the cost must be minimized.

As mentioned above, the present invention is not intended to be limited to a system or method which must satisfy one or more of any stated or implied object or feature of the invention and should not be limited to the preferred, exemplary, or primary embodiment(s) described herein. The foregoing description of a preferred embodiment of the invention has been presented for purposes of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as is suited to the particular use contemplated. All such modifications and variations are within the scope of the invention as determined by the claims when interpreted in accordance with breadth to which they are fairly, legally and equitably entitled.

The invention claimed is:

1. A safety needle assembly comprising:
a hub including:
a first end having a first engagement device for removably engaging with a reusable syringe;
a first needle extending outwardly from a second end; and
at least one engagement device disposed about an outer surface of said hub; and
a moveable sheath including a generally tubular shaped body having an inner diameter sized and shaped to fit over said outer surface of said hub and an outer surface of said reusable syringe and at least one engagement element that acts with said at least one engagement device to position said moveable sheath in at least one predefined position, wherein said moveable sheath is moveable along a longitudinal axis of said hub between an extended position whereby said moveable sheath is at least partially disposed about said hub and extends beyond a distal end of said first needle and a retracted position whereby said moveable sheath is substantially disposed about a barrel of said reusable syringe and said hub such that said first needle is substantially exposed.

2. The safety needle assembly as claimed in claim 1 wherein said first engagement device includes a threaded region adapted to threadably engage with said reusable syringe.

3. The safety needle assembly as claimed in claim 2 further including a second needle secured to said hub and extending outwardly from said hub in a direction substantially opposite said first needle, wherein said first engagement device includes an internally threaded cavity sized and shaped to substantially surround an outward most end of said second needle.

4. The safety needle assembly as claimed in claim 2 wherein said least one engagement device is selected from the group consisting of a biased tab and a biased ball.

5. The safety needle assembly as claimed in claim 1 wherein said at least one engagement element includes at least a first engagement element disposed proximate a distal end of said moveable sheath, wherein said first engagement element positions said moveable sheath in said extended position.

6. The safety needle assembly as claimed in claim 5 wherein said first engagement element includes at least one aperture.

7. The safety needle assembly as claimed in claim 5 wherein said first engagement element includes at least one recessed region.

8. The safety needle assembly as claimed in claim 5 wherein said at least one engagement element includes at least a second engagement element disposed proximate a proximal end of said moveable sheath, wherein said second engagement element positions said moveable sheath in said retracted position.

9. The safety needle assembly as claimed in claim 8 wherein said second engagement element includes at least one recessed region.

10. The safety needle assembly as claimed in claim 8 wherein said second engagement element includes at least one aperture.

11. The safety needle assembly as claimed in claim 1 wherein said hub and said movable sheath further include a protrusion and a channel disposed along a longitudinal axis thereof, wherein said protrusion and said channel define a predetermined path between said extended and said retracted positions along which said moveable sheath moves.

12. The safety needle assembly as claimed in claim 11 wherein said predefined path is substantially linear.

13. The safety needle assembly as claimed in claim 11 wherein said predefined path is substantially helical.

14. The safety needle assembly as claimed in claim 1 further including an end device adapted to be disposed proximate a distal end of said moveable sheath, wherein said end device substantially prevents access to said first needle when said needle assembly is disposed in said extended position.

15. The safety needle assembly as claimed in claim 14 wherein said end device includes a cap.

16. The safety needle assembly as claimed in claim 14 wherein said end device includes a pierceable membrane.

17. The safety needle assembly as claimed in claim 1 wherein moveable sheath further includes at least one finger gripper disposed about an outer surface of said moveable sheath proximate a proximal end.

18. A needle comprising:
a reusable syringe, said reusable syringe including:
a body portion defining an internal cavity sized and shape to contain a quantity of a medicament to be injected;
a first fastener disposed proximate a distal end of said body; and
a plunger disposed proximate a proximal end of said body, said plunger adapted to control the flow of said medicament; and
a needle guard assembly including
a hub having:
a first end having a second fastener for removably engaging with said first fastener of said reusable syringe;
a first needle extending outwardly from a second end; and
at least one engagement device disposed about an outer surface of said hub; and
a moveable sheath including a generally tubular shaped body having an inner diameter sized and shaped to fit over said outer surface of said hub and an outer surface of said reusable syringe and at least one engagement element that acts with said at least one engagement device to position said moveable sheath in at least one predefined position, wherein said moveable sheath is moveable along a longitudinal axis of said hub between an extended position whereby said moveable sheath is at least partially disposed about said hub and extends beyond a distal end of said first needle and a retracted position whereby said moveable sheath is substantially disposed about a barrel of said reusable syringe and said hub such that said first needle is substantially exposed.

19. The needle as claimed in claim 18 wherein said first and said second fastener includes a threaded connection and wherein said at least one engagement device includes a biased detent and said at least one engagement element includes at least one cavity disposed proximate a proximal end of said moveable sheath.

20. A needle comprising:
a reusable syringe, said reusable syringe including a body portion defining a cavity sized and shape to contain a quantity of a medicament to be injected and a plunger disposed proximate a proximal end of said body; and
a needle guard assembly including:
a hub having means for removably securing a first end of said hub to a distal end of said re-useable syringe;
a first needle extending outwardly from a second end;
a moveable sheath including a generally tubular shaped body having an inner diameter sized and shaped to fit over said outer surface of said hub and an outer surface of said reusable syringe;
means for positioning said movable sheath in at least one position, wherein said moveable sheath is moveable along a longitudinal axis of said hub between an extended position whereby said moveable sheath is at least partially disposed about said hub and extends beyond a distal end of said first needle and a retracted position whereby said moveable sheath is substantially disposed about a barrel of said reusable syringe and said hub such that said first needle is substantially exposed.
